SEQUENCE LISTING (21 sequences)

SEQ ID NO: 1 - protein, 208 aa, Artificial
  synthesized
QVQLQESGAELVKPGASVKISCKASGYTFTDHAIHWAKQKPEQGLEWIGYISPGNDDIKYNEKFKGKATLTADKSSSTAYMQLNSLTSEDSAVYFCKRSYYGHWGQGTTVTVSSGGGGSGGGGSGGGGSDIELTQSPAIMSASPGERVTMTCSASSSIRYIYWYQQKPGSSPRLLIYDTSNVASGVPFRFSGSGVWDLLFSHNQPNGG

SEQ ID NO: 2 - protein, 237 aa, Artificial
  synthesized
NSTMEWSWVFLFFLSVTTGVHSQVQLQQSDAELVKPGASVKISCKASGYTFTDHAIHWAKQKPEQGLEWIGYISPGNDDIKYNEKFKGKATLTADKSSSTAYMQLNSLTSEDSAVYFCKRSYYGHWGQGTTLTVSSGKTTPPVGGGGSGGGGSGGGGSDIELTQSPAIMSASPGERVTMTCSASSSIRYIYWYQQKPGSSPRLLIYDTSNVASGVPFRFSGSGVWDLLFSHNQPNGG

SEQ ID NO: 3 - protein, 238 aa, Artificial
  synthesized
TLTMEWSWVFLFFLSVTTGVHSQVQLQQSDAELVKPGASVKISCKASGYTFTDHAIHWAKQKPEQGLEWIGYISPGNDDIKYNEKFKGKATLTADKSSSTAYMQLNSLTSEDSAVYFCKRSYYGHWGQGTTLTVSSAKTTPPSVGGGGSGGGGSGGGGSDIELTQSPAIMSASPGERVTMTCSASSSIRYIYWYQQKPGSSPRLLIYDTSNVASGVPFRFSGSGVWDLLFSHNQPNGG

SEQ ID NO: 4 - protein, 262 aa, Artificial
  synthesized
MEWSWVFLFFLSVTTGVHSQVQLQQSDAELVKPGASVKISCKASGYTFTDHAIHWVKQNPEQGLEWIGYFSPGNDDFKYNERFKGKATLTADKSSSTAYVQLNSLTSEDSAVYFCTRSLNMAYWGQGTSVTVSSGGGGSGGGGSGGGGSDIVMSQSPSSLPVSVGEKVTLSCKSSQSLLYSGNQKNYLAWYQQKPGQSPKLLIYWASARESGVPDRFTGSGSGTDFTLSISSVKTEDLAVYYCQQYYSYPLTFGAGTKLVLK

SEQ ID NO: 5 - protein, 242 aa, Artificial
  synthesized
QVQLQESGAELVKPGASVKISCKASGYTFTDHAIHWAKQKPEQGLEWIGYISPGNDDIKYNEKFKGKATLTADKSSSTAYMQLNSLTSEDSAVYFCKRSYYGHWGQGTTVTVSSGGGGSGGGGSGGGGSDIVMSQSPSSLPVSVGEKVTLSCKSSQSLLYSGNQKNYLAWYQQKPGQSPKLLIYWASARESGVPDRFTGSGSGTDFTLSISSVKTEDLAVYYCQQYYSYPLTFGAGTKLVLK

SEQ ID NO: 6 - protein, 271 aa, Artificial
  synthesized
NSTMEWSWVFLFFLSVTTGVHSQVQLQQSDAELVKPGASVKISCKASGYTFTDHAIHWAKQKPEQGLEWIGYISPGNDDIKYNEKFKGKATLTADKSSSTAYMQLNSLTSEDSAVYFCKRSYYGHWGQGTTLTVSSGKTTPPVGGGGSGGGGSGGGGSDIVMSQSPSSLPVSVGEKVTLSCKSSQSLLYSGNQKNYLAWYQQKPGQSPKLLIYWASARESGVPDRFTGSGSGTDFTLSISSVKTEDLAVYYCQQYYSYPLTFGAGTKLVLK

SEQ ID NO: 7 - protein, 272 aa, Artificial
  synthesized
TLTMEWSWVFLFFLSVTTGVHSQVQLQQSDAELVKPGASVKISCKASGYTFTDHAIHWAKQKPEQGLEWIGYISPGNDDIKYNEKFKGKATLTADKSSSTAYMQLNSLTSEDSAVYFCKRSYYGHWGQGTTLTVSSAKTTPPSVGGGGSGGGGSGGGGSDIVMSQSPSSLPVSVGEKVTLSCKSSQSLLYSGNQKNYLAWYQQKPGQSPKLLIYWASARESGVPDRFTGSGSGTDFTLSISSVKTEDLAVYYCQQYYSYPLTFGAGTKLVLK

SEQ ID NO: 8 - protein, 244 aa, Artificial
  synthesized
AMGQVQLQQSDAELVKPGASVKISCKASGYTFTDHAIHWVKQKPEQGLEWIGYFSPGNGDIKYNEKFKGKATLTADKSSSTAYMQLNSLTSEDSAVYFCKRSYGNYDYWGQGTTLTVSSGGGGSGGGGSGGGGSDILLTQSPAILSVSPGERVSFSCRASQNIGTSIHWYQQRTNGSPRLLIKYASESVSGIPSRFSGSGSGTDFTLSINSVESEDIADYYCQHTNSWPTTFGGGTKLEIKLEH

SEQ ID NO: 9 - protein, 242 aa, Artificial
  synthesized
MGDILLTQSPAILSVSPGERVSFSCRASQNIGTSIHWYQQRTNGSPRLLIKYASESVSGIPSRFSGSGSGTDFTLSINSVESEDIADYYCQHTNSWPTTFGGGTKLEIKGGGGSGGGGSGGGGSQVQLQQSDAELVKPGASVKISCKASGYTFTDHAIHWVKQKPEQGLEWIGYFSPGNGDIKYNEKFKGKATLTADKSSSTAYMQLNSLTSEDSAVYFCKRSYGNYDYWGQGTTLTVSSLE

SEQ ID NO: 10 - protein, 453 aa, Clostridium tetani
LEKNLDCWVDNEEDIDVILKKSTILNLDINNDIISDISGFNSSVITYPDAQLVPGINGKAIHLVNNESSEVIVHKAMDIEYNDMFNNFTVSFWLRVPKVSASHLEQYGTNEYSIISSMKKHSLSIGSGWSVSLKGNNLIWTLKDSAGEVRQITFRDLPDKFNAYLANKWVFITITNDRLSSANLYINGVLMGSAEITGLGAIREDNNITLKLDRCNNNNQYVSIDKFRIFCKALNPKEIEKLYTSYLSITFLRDFWENPLRYDTEYYLIPVASSSKDVQLKNITDYMYLTNAPSYTNGKLNIYYRRLYNGLKFIIKRYTPNNEIDSFVKSGDFIKLYVSYNNNEHIVGYPKDGNAFNNLDRILRVGYNAPGIPLYKKMEAVKLRDLKTYSVQLKLYDDKNASLGLVGTHNGQIGNDPNRDILIASNWYFNHLKDKILGCDWYFVPTDEGWTND

SEQ ID NO: 11 - protein, 661 aa, Artificial
  synthesized
QVQLQESGAELVKPGASVKISCKASGYTFTDHAIHWAKQKPEQGLEWIGYISPGNDDIKYNEKFKGKATLTADKSSSTAYMQLNSLTSEDSAVYFCKRSYYGHWGQGTTVTVSSGGGGSGGGGSGGGGSDIELTQSPAIMSASPGERVTMTCSASSSIRYIYWYQQKPGSSPRLLIYDTSNVASGVPFRFSGSGVWDLLFSHNQPNGGLEKNLDCWVDNEEDIDVILKKSTILNLDINNDIISDISGFNSSVITYPDAQLVPGINGKAIHLVNNESSEVIVHKAMDIEYNDMFNNFTVSFWLRVPKVSASHLEQYGTNEYSIISSMKKHSLSIGSGWSVSLKGNNLIWTLKDSAGEVRQITFRDLPDKFNAYLANKWVFITITNDRLSSANLYINGVLMGSAEITGLGAIREDNNITLKLDRCNNNNQYVSIDKFRIFCKALNPKEIEKLYTSYLSITFLRDFWENPLRYDTEYYLIPVASSSKDVQLKNITDYMYLTNAPSYTNGKLNIYYRRLYNGLKFIIKRYTPNNEIDSFVKSGDFIKLYVSYNNNEHIVGYPKDGNAFNNLDRILRVGYNAPGIPLYKKMEAVKLRDLKTYSVQLKLYDDKNASLGLVGTHNGQIGNDPNRDILIASNWYFNHLKDKILGCDWYFVPTDEGWTND

SEQ ID NO: 12 - DNA, 20 bp, Artificial
  synthesized
tccatgacggtcctgagctc

SEQ ID NO: 13 - DNA, 20 bp, Artificial
  synthesized
atcgactcctgagcgttctc

SEQ ID NO: 14 - DNA, 18 bp, Artificial
  synthesized
ccatggactggacctgga

SEQ ID NO: 15 - DNA, 20 bp, Artificial
  synthesized
ccatggagtttgggctgagc

SEQ ID NO: 16 - DNA, 20 bp, Artificial
  synthesized
atggacatactttgttccac

SEQ ID NO: 17 - DNA, 20 bp, Artificial
  synthesized
atgaaacacctgtggttctt

SEQ ID NO: 18 - DNA, 20 bp, Artificial
  synthesized
atggggtcaaccgccatcct

SEQ ID NO: 19 - DNA, 20 bp, Artificial
  synthesized
atgtctgtctccttcctcat

SEQ ID NO: 20 - DNA, 20 bp, Artificial
  synthesized
cagtaatacacggccgtgtc

SEQ ID NO: 21 - DNA, 24 bp, Artificial
  synthesized
acctgaggagacggtgaccagggt

(19) United States
(12) Patent Application Publication
(10) Pub. No.: US 2003/0212027 A1
(43) Pub. Date: Nov. 13, 2003

(54) Vaccine formulations and methods for immunizing an individual against shed antigen specific B cells

(75) Inventors: Emilio Barbera-Guillem, Powell, OH (US); M. Bud Nelson, Worthington, OH (US)

Correspondence Address: BENESCH, FRIEDLANDER, COPLAN & ARONOFF LLP, ATTN: IP DEPARTMENT DOCKET CLERK, 2300 BP TOWER, 200 PUBLIC SQUARE, CLEVELAND, OH, 44114, US

(73) Assignee: BioCrystal, Ltd., Westerville, OH

(21) Appl. No.: 10/336,210

(22) Filed: Jan. 3, 2003

Related U.S. Application Data
(63) Continuation of application No. 09/594,985, filed on Jun. 15, 2000, now abandoned.
(60) Provisional application No. 60/139,521, filed on Jun. 16, 1999.

(51) Int. Cl. A61K 48/00

(52) U.S. Cl. 514/44

(57) ABSTRACT

Provided are methods for inducing an immune response reactive with idiotypes on shed antigen-specific B cells in an individual by administering an immunologically effective amount of a vaccine formulation. Also provided are vaccine formulations comprising one or more peptides, wherein a peptide comprises an idiotype of an antibody that binds to an epitope of shed antigen; or one or more polynucleotides, wherein a polynucleotide encodes a peptide comprising an idiotype of an antibody that binds to an epitope of shed antigen.

This non-provisional application is a continuation of application Ser. No. 09/594,985, filed Jun. 15, 2000, which is a non-provisional application based on earlier provisional application Serial No. 60/139,521, filed Jun. 16, 1999, now abandoned, which is herein incorporated by reference.

### FIELD OF THE INVENTION

The present invention is related to novel compositions and methods for treating an individual having one or more specific subpopulations of B lymphocytes. More particularly, the present invention is related to a vaccine for use to deplete B cells which express surface immunoglobulin with idiotypes and which may be involved in immune complex-mediated disease progression in an organ-specific autoimmune disease.

### BACKGROUND OF THE INVENTION

1. Solid, Nonlymphoid Tumors.

The response of an individual to nonlymphoid tumor cells involves the reactions and counteractions mediated by both cellular and humoral arms of the immune system. Non-lymphoid tumor cell growth may represent a disturbance in the equilibrium of the immune system that is pre-existing, and/or induced by the tumor cells themselves. However, most investigations to date have focused on the role of T cells in tumor immunity. In lymphoid tissue regional to tumor of a solid, nonlymphoid tumor-bearing individual, there is often an increased number of immune cells that include B lymphocytes (B cells). Similarly, the number of B cells increase in the regional lymph nodes concomitantly with tumor development. It is believed that such B cells appear to be able to elicit anti-tumor immunity. The role of the B cells in the host response to tumor, and the tumor-associated antigens recognized by B cells, remain poorly defined.

However, the present inventors have discovered that B cells can be induced by shed tumor antigen which results in a pro-tumor immune response which can promote tumor progression, as described in more detail in co-pending application Ser. No. 09/411,116, the disclosure of which is herein incorporated by reference. More particularly, the B cells which are activated against shed tumor antigen which are found in a growing tumor are generally shed antigen-specific B cells involved in a pro-tumor immune response. Thus, for individuals having a pro-tumor immune response, there is a need for depleting B cells which express surface immunoglobulin with idiotypes which may, when part of antibody secreted by plasma cells, be involved in immune complex-mediated disease progression comprising the pro-tumor immune response.

2. Multiple Sclerosis

Multiple sclerosis (“MS”) is an organ-specific autoimmune disease of the central nervous system. MS affects 250,000 to 350,000 in the United States, and approximately 1 million people worldwide. There is no clear understanding of the immunopathogenic processes associated with MS; and, to date, there lacks published evidence of a unique immunologic abnormality in individuals with MS. Because of the incomplete understanding of the pathogenesis of MS, therapeutic advances have been slow to emerge. The main focus on pathogenesis of MS has been on autoreactive T cells which, when activated and reach the central nervous system, are thought to secrete proinflammatory cytokines. These cytokines are believed to induce astrocytes and leukocytes (including by activating microglia and macrophages) to secrete enzymes which damage myelin, and result in inflammation, demyelination, and axonal damage in the central nervous system characteristic in MS. However, humoral immune responses against peptide epitopes of MBP and MOG have been shown to cause demyelination both in vivo and in vitro.

Thus, for individuals with MS, there is a need for therapies which can prevent relapses, or inhibit the progressive worsening of the disease, or can in some way interfere with the disease progression process.

3. Id Vaccines

The sIg of malignant B cells of each B cell tumor (e.g., lymphoma or myeloma) display unique antigenic determinants within the variable regions of the Ig heavy and light chains. Since the B cell malignancy is monoclonal, all tumor cells of each lymphoma produce the same Ig protein. Thus, the idiotype of the surface Ig of the clonally-derived malignant B cells in a given lymphoma represents a unique tumor-specific antigen that can generally be distinguished from the Ig on normal (nonmalignant) B cells. Hence, tumor-specific idiotype (Id) vaccines have been used in the treatment of patients with B-cell lymphoma, including in Phase I/II trials. Induction by the Id vaccine of anti-idiotypic antibodies alone are capable of inducing cell death, presumably through the direct induction of apoptosis, or indirectly through complement fixation or antibody dependent cellular cytotoxicity mechanisms. Also, it is believed that cell-mediated immune responses (e.g., cytotoxic T cells against the lymphoma cells) may be induced, which can also play a role in the beneficial clinical outcome resulting from Id vaccination.

In one clinical trial of patients with non-Hodgkin's lymphoma, an Id vaccine was produced by isolating the Ig protein from the tumor cells of each patient, and then conjugating the respective Ig protein to carrier protein keyhole limpet hemocyanin (KLH). Those patients who generated specific immune responses against the idiotypes of their tumor Ig had significantly prolonged duration of freedom from disease progression and overall survival. In another clinical trial, autologous dendritic cells were pulsed with the Ig (idiotypic) protein, and patients with follicular B-cell lymphoma were vaccinated with the vaccine containing their respective idiotype. All of the patients receiving the vaccine developed measurable antitumor immune responses against their B cell tumors. The majority of these vaccinated patients had clinically beneficial responses, ranging from complete B cell tumor regression, partial B cell tumor regression, or resolving of all evidence of the lymphoma.

Using standard nucleic acid amplification techniques, the idiotypic immunoglobulin V genes were isolated from a B cell lymphoma patient. An expression vector, constructed to encode a lymphoma-derived idiotypic single chain Fv (scFv), was injected intramuscularly into the patient in a vaccine protocol. In some cases, only a low amount of anti-idiotypic antibody was induced. Hence, in efforts to enhance antibody induction, similar vaccines have been produced in which the scFv genes (V_{H} and V_{L} genes) were fused to DNA encoding fragment C of tetanus toxin (“FrC”). It has been reported that for DNA vaccines produced from scFv (from B cell tumors of lymphoma patients and of myeloma patients) fused to FrC resulted in significant (e.g., approximately 50 fold) promotion of antibody as compared to scFv alone. Therefore, it was concluded that (a) such DNA vectors can survive in vivo, can enter cells, and can undergo appropriate expression (e.g., transcription, translation, and presentation of the idiotype to the immune system) in vivo in a manner sufficient to generate a therapeutically effective amount which can induce an immune response of apparent therapeutic benefit; and (b) scFv can maintain proper folding, when fused with FrC, for inducing an immune response against the respective idiotype.

### SUMMARY OF THE INVENTION

Accordingly, it is a primary object of the present invention to provide vaccine formulations and methods for depleting shed antigen-specific B cells, wherein the B cells may differentiate into plasma cells that secrete antibody having binding for shed antigen.

It is another object of the present invention to provide vaccine formulations for treating immune complex-mediated disease progression in organ-specific autoimmune diseases excaberated by a humoral immune response against one or more epitopes expressed on shed antigen, wherein such organ-specific autoimmune diseases may include, but are not limited to, cancers (solid, nonlymphoid tumors), and multiple sclerosis.

It is a further object of the present invention to provide vaccine formulations for treating organ-specific autoimmune diseases which are exacerbated by plasma cell production of antibodies against one or more epitopes of shed antigen, wherein shed antigen-specific B cells serve as a source of such plasma cells.

It is another object of the present invention to provide a vaccine formulation comprising an idiotype (Id) comprising idiotopes found on surface immunoglobulin (sIg) of shed antigen-specific B cells, wherein an idiotope comprises an antigenic determinant present in the unique antigen recognition site of a sIg having binding specificity for an epitope of the shed antigen.

It is further object of the present invention to provide a vaccine formulation comprising an expression vector or a plurality of expression vectors, with each expression vector encoding a peptide comprising an Id found on the sIg of shed antigen specific B cells; and wherein the vector is capable of expressing the peptide in, and the peptide is capable of being secreted or released from, mammalian cells containing the vector.

It is further object of the present invention to provide a vaccine formulation comprising an expression vector or a plurality of expression vectors, wherein each expression vector encodes a recombinant peptide comprising an Id of sIg having binding specificity for an shed antigen epitope, wherein a recombinant peptide further comprises an amino acid sequence for enhancing the immune response, and wherein the vector is capable of expressing the recombinant peptide in, and the recombinant peptide is capable of being secreted or released from, mammalian cells containing the vector.

It is another object of the present invention to provide a method of inducing an immune response reactive with idiotypic determinants comprising antigenic determinants present in the unique antigen recognition sites of sIg on shed antigen-specific B cells, wherein the method comprises administering to an individual an immunologically effective amount of a vaccine formulation comprising one or more peptides, or one or more expression vectors encoding and capable of expressing a peptide.

A further object of the present invention is to provide a method for producing sequences encoding idiotypic determinants (idiotypes) of shed antigen-specific B cells which have sIg having binding specificity for an epitope for shed antigen.

The foregoing objects are achieved by identifying a novel mechanism by which a shed antigen, released from a process initiated at a site of an organ, induces (activates) a subpopulation of B cells in a humoral immune response that may promote immune complex-mediated disease progression. The activated B cells may then proliferate, and/or differentiate into plasma cells which secrete antibody against the shed antigen (“anti-shed antigen antibody”). In a preferred embodiment, the antibody has binding specificity for one or more terminal carbohydrate epitopes of shed antigen. In a more preferred embodiment, the antibody has binding specificity for an epitope comprising a terminal, alpha 2,6-linked sialic acid. The shed antigen and anti-shed antigen antibody form complexes which may activate host cells in an autoimmune process which results in the promotion of tissue destruction, and progression of the organ-specific autoimmune disease. In one embodiment of the present invention, administered is an immunologically effective amount of a vaccine formulation comprising an idiotype vaccine (Id vaccine) to an individual having shed antigen-specific B cells which have sIg having binding specificity for shed antigen. In a preferred embodiment, the sIg has binding specificity for an epitope comprising a terminal, alpha 2,6-linked sialic acid. In another embodiment, to an individual having an organ-specific autoimmune disease is administered an immunologically effective amount of a vaccine formulation comprising an Id vaccine, wherein the Id comprises an antigenic determinants present in the unique antigen recognition sites of the sIg of shed antigen-specific B cells. In a preferred embodiment, the sIg has binding specificity for an epitope comprising a terminal, alpha 2,6-linked sialic acid. In a preferred embodiment, the vaccine formulation may induce an immune response which comprises anti-idiotypic antibody in an immunologically effective amount to cause depletion of the shed antigen-specific B cells in the immunized individual. The vaccine formulation may induce an immune response which may also function by additional mechanisms to inhibit immune complex-mediated disease progression in the organ-specific autoimmune disease.

The above and other objects, features, and advantages of the present invention will be apparent in the following Detailed Description of the Invention when read in conjunction with the accompanying drawings.

### BRIEF DESCRIPTION OF THE DRAWINGS

FIG. 1 is a bar graph illustrating in vivo spleen tumor cell growth and liver metastasis (combined score) in the presence of splenic B lymphocytes from tumor bearing mice (T-SpL), B lymphocytes from tumor (B-TIL), and splenic B lymphocytes from normal mice (N-Spl).

FIG. 2 is a bar graph illustrating invasion of shed tumor antigen-secreting tumor cells through matrix when incubated with various cellular components, antibodies, or antibody fragments.

### DETAILED DESCRIPTION OF THE PREFERRED EMBODIMENTS

Definitions

The term “B cells” is used herein, for purposes of the specification and claims, to mean mammalian (and preferably human) nonmalignant B cells. As known to those skilled in the art, malignant B cells refers to cancer cells of B cell origin, such as B cell lymphomas, myelomas, and B cell leukemias. Thus, the term “B cells”, as used herein (a) specifically excludes B cell lymphomas, B cell leukemias, B cell myelomas, and cancer cells of B cell origin; and (b) comprises B cells (e.g., memory B cells or mature B cells) (i) which have been stimulated by a shed antigen (in forming “shed antigen-specific B cells”), (ii) which have a cell surface-bound immunoglobulin (sIg) comprising antibody having binding specificity for an epitope of shed antigen, and (iii) which can be induced to proliferate, and/or differentiate into plasma cells which produce/secrete antibody having binding specificity for an epitope of shed antigen. In a preferred embodiment, the epitope to which the antibody has binding specificity is an epitope selected from the group consisting of Tn antigen, an epitope of shed carcinoembryonic antigen (CEA), a terminal, alpha 2,6-linked sialic acid, and a combination thereof. In a preferred embodiment, such antibody can complex with shed antigen in forming immune complexes that may contribute to a promotion of an organ-specific autoimmune disease by immune complex-mediated mechanisms, as will be more apparent from the following embodiments. In a preferred embodiment, these shed antigen-specific B cells are located substantially at the site of chronic inflammation in the organ affected by the organ-specific autoimmune disease, and/or in lymphoid tissue containing shed antigen which is proximal to the organ affected by the organ-specific autoimmune disease.

The term “solid, nonlymphoid tumor” is used herein, for purposes of the specification and claims, to mean any tumor (a) of ductal epithelial cell origin, including tumors originating in an organ such as liver, lung, brain, bone marrow, adrenal gland, breast, colon, pancreas, stomach, prostate, gastrointestinal tract, or reproductive tract (cervix, ovaries, endometrium etc.), or metastases thereof; and (b) which secretes or produces shed antigen (e.g., serous, or endometroid, or mucinous tumors). For the purposes of the present invention, “solid, non-lymphoid tumor” may also include melanoma. In a preferred embodiment, the solid, nonlymphoid tumor produces shed antigen having one or more epitopes comprising terminal, alpha 2,6-linked sialic acid.

The term “immune complex-mediated disease progression” is used herein, for purposes of the specification and claims, to mean one or more mechanisms for inducing a pathology of an organ-specific autoimmune disease which may be, at least in part, promoted by immune complex activation of immune effector cells, wherein the activated immune effector cells contribute to the pathology such as by causing one or more of tissue degradation, inflammation, and angiogenesis; wherein the immune complexes involved in the organ-specific autoimmune disease are comprised of shed antigen, and anti-shed antigen antibody. In a preferred embodiment, the anti-shed antigen antibody has binding specificity for an epitope comprising a terminal, alpha 2,6-linked sialic acid.

The term “organ-specific autoimmune disease” is used herein, for purposes of the specification and claims, to refer to a disordered state of immunological regulation which contributes to pathogenesis in an affected organ; and may include a humoral immune response against one or more epitopes expressed by shed antigen. The disordered state of immunological regulation may affect an organ selected from the group consisting of the central nervous system, the peripheral nervous system, an organ in which solid, nonlymphoid tumor is present (e.g., breast, lung, colon, pancreas, liver, stomach, prostate, gastrointestinal tract, or reproductive tract), or the pancreas. The organ-specific autoimmune disease may include, but is not limited to, multiple sclerosis, and a primary solid, nonlymphoid tumor or its metastases. An example of such a humoral immune response against one or more epitopes of shed antigen is a humoral immune response against one or more epitopes comprising a terminal, alpha 2,6-linked sialic acid; such as observed in a pro-tumor immune response, as described herein in more detail.

The term “peptide” is used herein, for purposes of the specification and claims, to mean an idiotype comprising an antigenic determinant present in the unique antigen recognition site of sIg of shed antigen-specific B cells having binding specificity for an epitope expressed on shed antigen. In a preferred embodiment, the peptide may comprise from about 50 amino acids to about 1500 amino acids. In a most preferred embodiment, the peptide comprises a variable heavy chain region (known to those skilled in the art as “V_{H}”) linked to a variable light chain region (known to those skilled in the art as “V_{L}”) by use of an linker known in the art (e.g., comprising from about 2 to about 50 amino acids). As apparent to one skilled in the art, the order in which the V_{H} is linked to V_{L} may be varied (eg., one or more of V_{H}-V_{L} or V_{L}-V_{H} may provide either the correct or optimal folding of the peptide for induction of an immune response comprising anti-idiotypic antibody), depending on factors which include, but are not limited to, the binding site of the idiotype (e.g., for binding to the epitope), and any degree of conformation thereof which relates to immunogenicity. Thus, produced from an antibody having binding specificity for an epitope of shed antigen is an amino acid sequence comprising a V_{H} region and an amino acid sequence comprising a V_{L} region which are linked together by a linker. For example, SEQ ID NO:1 comprises a V_{H} (comprising amino acids 1 to 114) linked by a linker (comprising amino acids 115 to 129) to a V_{L} (comprising amino acids 130 to 208). As apparent to one skilled in the art, the V_{H} of a first antibody may be linked to the V_{L} of a second antibody, wherein each antibody has binding specificity for an epitope of shed antigen (e.g., where the epitope comprises a terminal alpha 2,6-linked sialic acid; see, for example, SEQ ID NO:2). In one embodiment, the vaccine formulation comprises a plurality of peptides representing a plurality of idiotypes from shed antigen-specific B cells present in the organ-specific autoimmune disease. In another embodiment, the vaccine formulation comprises a peptide comprising a single idiotype, such as comprising an antigenic, and contiguous sequence of amino acid residues which form a binding domain having binding specificity for an epitope comprising a terminal, alpha 2,6-linked sialic acid. In a preferred embodiment, the peptide comprises the idiotype comprising a V_{H} linked to V_{L}, and further comprises an immunostimulatory sequence (“ISS”) operably linked thereto. The order of the peptide can be varied; i.e., may be selected from the group consisting of V_{H}-V_{L}-ISS, V_{L}-V_{H}-ISS, ISS-V_{H}-V_{L}, ISS-V_{H}-V_{L}, and a combination thereof. In a preferred embodiment, the peptide forms a binding region having binding specificity for the sTn antigen. Binding specificity can be measured using standard techniques known in the art (e.g., ELISA). In a preferred embodiment, the peptide may comprise an amino acid sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, or a peptide having an amino acid sequence that varies by less than 10% of any one of SEQ ID NOs: 1-9, and is capable of inducing anti-idiotype antibody against an antibody having binding specificity for an epitope of shed antigen. In an alternative embodiment, a vaccine formulation may comprise the peptide comprising multiple copies of the idiotype (e.g., V_{H}-V_{L}-V_{L}-V_{H}) . By operatively coupling more than one monomer of the peptide together, the vaccine formulation may be sufficiently immunogenic so as to obviate any need to fuse the peptide with an immunostimulatory sequence to enhance immunogenicity. Operatively coupling (e.g., conjugating, or fusing by recombinant means) a peptide to an immunostimulatory sequence may render the peptide more immunogenic, in forming a vaccine formulation. Suitable immunostimulatory sequences are known to those skilled in the art to include, but are not limited to, key-hole limpet hemocyanin (KLH), heat shock proteins, tetanus toxin fragment C (FrC), soybean trypsin inhibitor, hepatitis B surface antigen, diphtheria toxin, beta-galactosidase, and the like. Preferred immunostimulatory sequences may comprise immunogenic determinants from vaccines commonly administered to humans (e.g., hepatitis B surface antigen, tetanus toxin, diphtheria toxoid, rubeola, varicella, Haemophilus influenzae, and the like) so as to act as a booster of immunity. Operatively coupling a peptide to an immunostimulatory sequence can be achieved using methods known in the art, the exact choice of which will depend on the nature of the peptide, and the nature of the immunostimulatory sequence to be coupled. These methods include, but are not limited to, use of a bifunctional agent or derivatizing agent; or genetically engineering the peptide to be co-expressed with the immunostimulatory sequence operatively coupled thereto. For example, an amino acid sequence comprising FrC comprises SEQ ID NO:10. In a preferred embodiment, a peptide was recombinantly produced to comprise SEQ ID NO:1 and SEQ ID NO:10 as shown by SEQ ID NO:11. Similarly, any one of SEQ ID NOs. 2-9 may also be operatively coupled to SEQ ID NO:10.

“Consisting of”, in relation to amino acid sequence of a peptide described herein, is a term used hereinafter for the purposes of the specification and claims to refer to a conservative substitution or modification of one or more amino acids in that sequence such that the tertiary configuration of the peptide in comprising an idiotype of antibody having binding specificity for an epitope of shed antigen, is substantially unchanged (i.e., is still capable of inducing an immune response comprising anti-idiotypic antibody against an antibody having binding specificity for an epitope of shed antigen). In a preferred embodiment, the amino acid sequence of the peptide (e.g., any one of SEQ ID NOs:1-9) may be substituted by 10% or less and remain capable of inducing the anti-idiotype antibody when used in a vaccine formulation according to the present invention. “Conservative substitutions” is defined by aforementioned function, and includes substitutions of amino acids having substantially the same charge, size, hydrophilicity, and/or aromaticity as the amino acid replaced. Such substitutions known to those of ordinary skill in the art include, but are not limited to, glycine-alanine-valine, isoleucine-leucine, tryptophan-tyrosine, aspartic acid-glutamic acid, arginine-lysine, asparagine-glutamine, and serine-threonine. Such conservative substitutions would not be expected to substantially alter the biological activity of the peptide such that it can no longer be used to induce an immune response compris-ing anti-idiotype antibody against an antibody having binding specificity for an epitope of shed antigen. “Modification”, in relation to amino acid sequence of a peptide, is defined functionally as a deletion of or addition of one or more amino acids which does not impart a substantial change in the biological activity or specificity of the peptide such that it can no longer be used to induce an immune response comprising anti-idiotype antibody against an antibody having binding specificity for an epitope of shed antigen. In particular, a peptide according to the present invention may be modified, using methods known by those skilled in the art, by adding one or more amino acids or functional groups for one or more properties which include, but are not limited to, immunostimulatory sequences for enhancing the immunogenicity of the peptide, and to minimize toxicity, if any.

“Consisting of”, in relation to a nucleic acid sequence or polynucleotide described herein, is a term used hereinafter for the purposes of the specification and claims to refer to substitution of nucleotides as related to third base degeneracy. As appreciated by those skilled in the art, because of third base degeneracy, almost every amino acid can be represented by more than one triplet codon in a coding nucleotide sequence. Further, minor (e.g., less than 10% of the nucleic acid sequence) base pair changes may result in variation (conservative substitution) in the amino acid sequence encoded, are not expected to substantially alter the biological activity of the product encoded by the polynucleotide. Thus, a nucleic acid sequencing encoding a peptide as disclosed herein, may be modified slightly in sequence (e.g., substitution of a nucleotide in a triplet codon), and yet still encode its respective gene product of the same amino acid sequence.

The term “shed antigen” is used herein, for purposes of the specification and claims, to mean a glycomolecule (glycoprotein or glycolipid or carbohydrate-containing molecule) which:

(a) by itself, or in an aggregated or oligomeric (e.g., two or more monomers which are associated with each other by a covalent or noncovalent or other force which mediates contact between the monomers) form, has a molecular size equal to or greater than about 100 kilodaltons;

(b) is released (e.g., shed) during the immune complex-mediated disease process of an organ-specific autoimmune disease, thereby becoming soluble and allowing movement to reach B cells which are local, regional or distal to the organ affected by the organ-specific autoimmune disease process;

(c) comprises one or more immunogenic epitopes;

(d) is capable of inducing a humoral immune response resulting in the production and secretion of antibody (anti-shed antigen antibody) which has binding specificity for an epitope of shed antigen, wherein the antibody may predominately be of an IgG class; and

(e) can interact with anti-shed antigen antibody in forming immune complexes, wherein the immune complexes may bind and cross-link Fc receptors (e.g., FcγRI) present on the surface of Fc receptor-expressing cells.

For solid, nonlymphoid tumors, the shed antigen comprises shed tumor antigen which is exemplified by mucin and mucin-like molecules (e.g., CEA). Briefly, mucins are high molecular weight glycoproteins (e.g., greater than about 100 kiloDaltons (kD) in molecular mass) of which a significant portion of the polypeptide backbone comprises a domain composed of tandomly repeated peptide subunits (e.g. about 20 to about 125 repeats). Mucins are found on normal ductal epithelial cells in sequestered locations that are not normally exposed to the immune system (e.g., restricted to the lumen of duct). However, in processes such as transformation (e.g., pre-cancerous) or tumor development, and due to various factors (e.g., the increased production of mucin, lack of availability of glycosyltransferases), tumor cells produce and secrete mucin in a form of altered glycosylation such that expressed is a repeated epitope comprising terminal, alpha-linked carbohydrate epitopes. Such epitopes are exemplified by a terminal, alpha 2,6-linked sialic acid; and Tn antigen (a terminal alpha-linked galactosamine (GalNAc), as known to those skilled in the art). Thus, because of the altered glycosylation in growing tumors, the shed tumor mucin has one or more terminal epitopes not normally found on mucin, wherein such terminal epitopes can induce a humoral immune response against the shed antigen (shed tumor mucin).

Preferably such epitopes on the glycomolecule may comprise a terminal N-acetylneuraminic acid (“Neu5Ac” or “NeuAc”) alpha 2,6-linked to either a galactosamine (GalNAC) or galactose (Gal); or may comprise a terminal N-glycolylneuraminic acid (“Neu5Gc” or “NeuGc”, which comprises Neu5Ac to which is added a single oxygen atom) alpha 2,6-linked to either a galactosamine (GalNAC) or galactose (Gal); or Tn. Thus, preferably the epitope may be selected from the group consisting of Tn (substantially comprising a terminal alpha, linked GalNAc), sialyl Tn (sTn) antigen (substantially comprising the terminal NeuAc portion of NeuAcα2→6GalNAcα1→), or other epitope comprising a terminal alpha 2,6-linked sialic acid (e.g., substantially comprising a terminal NeuAc on the carbohydrate chains comprising (a) NeuAcα2→6Gal→, (b) NeuAcα2→6Galβ1→4GlcNAc→ (e.g., as found on CEA shed by adenocarcinomas), (c) NeuAcα2→6Galβ1→; or substantially comprising a terminal NeuGc on the carbohydrate chains comprising (a) NeuGcα2→6Gal→, (b) NeuGcα2→6GalNAc α1→, (c) NeuGcα2→6Galβ1→), and a combination thereof (e.g., having a mixture of epitopes comprising terminal alpha-linked carbohydrate residues). An example of a mucin-like glycoprotein which is differentially glycosylated by tumor cells, and is shed by tumor cells, is SSEA-1 antigen. Examples of shed antigens comprising glycolipids shed in organ-specific autoimmune diseases include, but are not limited to, GDlα, GTlaα, and GQlbα. For purposes of illustration, and not limitation, in a preferred embodiment of the present invention wherein the organ-specific autoimmune disease comprises a solid, nonlymphoid tumor, the shed antigen comprises the gene product of the MUC-1 gene (also known as polymorphic epithelial mucin), shed CEA, or a combination thereof.

The term “individual” is used herein, for purposes of the specification and claims, to mean a mammal, and preferably a human; and more preferably a human individual who is at risk (e.g., environmentally and/or genetically) of developing, or has developed, an organ-specific autoimmune disease. For example, an individual at risk of developing, or has developed, an organ-specific autoimmune disease promoting nonlymphoid tumor progression may include an individual having a primary tumor comprising a solid, non-lymphoid tumor and/or its metastases; an individual with a pre-cancerous condition comprising transformed (abnormal in proliferation and/or genetic makeup as compared to normal epithelial cells of the same type) cells of ductal epithelial origin which release shed tumor antigen; an individual who is at high risk for developing a solid, non-lymphoid tumor; an individual who has developed a pro-tumor immune response, or an individual who has been treated for a solid, nonlymphoid tumor and thereby inherently carries a risk of recurrence. Similarly, an individual at risk of developing an organ-specific autoimmune disease comprising secondary progressive multiple sclerosis is an individual who presents with relaxing remitting multiple sclerosis. Thus, a method of inducing an immune response reactive with shed antigen-specific B cells in an individual comprises administering the vaccine formulation to an individual who is at risk of developing, or has developed, an organ-specific autoimmune disease.

The term “immune complexes” is used herein, for purposes of the specification and claims, to mean complexes comprising anti-shed antigen antibody complexed with shed antigen for which it has binding specificity. In a preferred embodiment, the antibody is of an IgG subtype, and more preferably, an IgGl subtype. In a preferred embodiment, the antibody has binding specificity for an epitope comprising a terminal, alpha-linked carbohydrate expressed by shed antigen, wherein such epitope is not expressed by normal (undiseased) human tissue so as to be exposed to the immune system. Preferably, such immune complexes are immunostimulatory immune complexes which have a threshold level for spacing and number of antibody molecules necessary for cell surface receptor (Fc receptor; e.g., FcγRI) crosslinking. Immune complex crosslinking of immune effector cells which are mediators of inflammation and/or angiogenesis (e.g., one or more of granulocytes, macrophages, vascular endothelial cells, microglia, astrocytes) may induce a cascade of inflammatory processes which promote the development and/or the exacerbation (individually or collectively referred to herein as “progression”) of an immune complex-mediated disease. For example, and in an embodiment in which the shed antigen is shed tumor antigen, the resultant immune complexes may mediate tumor progression (e.g., promoting one or more of tumor growth, invasion, and metastasis) by one or more mechanisms such as inducing a cascade of inflammatory processes which promote tumor progression such as release of molecules which participate in tissue destruction; inhibiting tumor cell-associated antigen presentation to human tumor-specific cytotoxic lymphocytes; increasing expression on tumor cells of cell-surface molecules which promote metastasis; crosslinking Fc gamma receptors on tumor cells which may induce tumor proliferation and/or an increase in tumor production of shed tumor mucin; and interacting with and binding to endothelial cells in promoting angiogenesis.

The term “depletion” is used herein in reference to B cells nd more preferably B cells comprising shed antigen-specific B cells, and for purposes of the specification and claims, to mean one or more of: blocking of B cell function; functional inactivation of B cells; cytolysis of B cells; inhibiting the proliferation of B cells; inhibiting the differentiation of B cells to plasma cells; causing a B cell dysfunction which results in a therapeutic benefit; and reduction in the number of B cells. B cell depletion may be a result of one or more mechanisms including, but not limited to, clonal inactivation, apoptosis, antibody-dependent cellular cytotoxicity, complement-mediated cytotoxicity, a signal pathway mediated inactivation, or dysfunction or cell death, and cell-mediated cytotoxicity.

The term “immunologically effective” is used herein in reference to the vaccine and method according to the present invention, and for purposes of the specification and claims, to mean administering an amount (e.g., a dose and of a specificity sufficient) to effect one or more of: a depletion of B cells, and a reduction (e.g, in the amount and/or function) of immunostimulatory immune complexes in the treated individual. Reduction of such immunostimulatory immune complexes may reduce interaction with immune effector cells that are activated by such immune complexes to produce mediators of inflammation involved in the progression of the organ-specific autoimmune disease; and may interfere with shed antigen presentation (as part of immune complexes) by antigen presenting cells (e.g., dendritic cells or antigen presenting B cells) to naive B cells.

The terms “immunizing” and “immunization”, when used in reference to the vaccine and method according to the present invention, and for purposes of the specification and claims, mean prophylatically or therapeutically. Thus, the vaccine of the present invention may be used to induce an immune response for protecting an individual from occurrence of (e.g., one at high risk for developing) the organ-specific autoimmune disease (“prophylactic”), or may be used to induce an immune response in an individual already having developed the organ-specific autoimmune disease (“therapeutic”).

The terms “immune complex-mediated disease progression”. is used herein, for purposes of the specification and claims to mean the promotion of a disease process by immune complexes comprising shed antigen and an antibody induced against shed antigen. In a preferred embodiment, the antibody has binding specificity for an epitope comprising a terminal, alpha-linked carbohydrate (e.g., a terminal, alpha 2,6-linked sialic acid). Preferably, the antibody is IgG. Such immune complexes may promote disease progression by one or more mechanisms including, but not limited to: binding and cross-linking Fc receptors (FcR; e.g., FcγRI) on immune effector cells resulting in the release of inflammatory mediators which promote local tissue destruction and angiogenesis. For example, immune complexes may promote demyelination in MS, or may promote connective tissue destruction in facilitating invasion of tumor cells in solid, nonlymphoid tumor, or may promote destruction of islet cells in the pancreas.

In its broadest aspect, the present invention is based on the discovery that shed antigens, produced in a process of an organ-specific autoimmune disease, can comprise one or more terminal epitopes capable of inducing a humoral immune response that results in the production of antibody against the shed antigen; and that immune complexes formed between this antibody and the shed antigen for which it has binding specificity are capable of promoting progression of the organ-specific autoimmune disease. The present invention is also based on the discovery that antibodies, comprising idiotypes expressed on shed antigen-specific B cells, may be involved in the promotion of an immune complex-mediated disease; and that idiotypes expressed on B cells involved in the promotion of immune complex-mediated disease progression of an organ-specific autoimmune disease may be shared or may be cross-reactive with idiotypes expressed on B cells involved in the promotion of other organ-specific autoimmune diseases.

The idiotype comprises a distinctive antigenic determinant in the variable or idiotypic region of the antibody molecule. Thus, in individuals having an immune complex-mediated disease, there exists clonally-derived, shed antigen-specific B cells, wherein such B cells have as part of their surface Ig an idiotype that represents a unique and specific antigen that can generally be distinguished from the Ig on most B cells which are not related to the organ-specific autoimmune disease process. Further, the shed antigen-specific B cells can be concentrated in the anatomical area of the organ-specific autoimmune disease process. Accordingly, the invention provides for (a) the production of a vaccine formulation from one or more idiotypes of shed antigen-specific B cells; (b) a vaccine formulation which, when administered to an individual in an immunologically effective amount, induces an effective immune response to cause depletion of B cells comprising shed antigen-specific B cells in the immunized individual, and which may function by additional mechanisms to inhibit the progression of the organ-specific autoimmune disease. In one embodiment of the present invention, to an individual is administered an immunologically effective amount of the vaccine formulation composition comprising an idiotype vaccine (Id vaccine comprised of one or a plurality of idiotypes), wherein an idiotype comprises an antigenic determinant present in the unique antigen recognition site of antibody having binding specificity for an epitope of shed antigen. In a preferred embodiment, the epitope comprises a terminal, alpha-linked carbohydrate, as exemplified by an epitope comprising a terminal, alpha 2,6-linked sialic acid. The Id vaccine may result in the induction of an immune response which comprises production of anti-idiotypic antibody in sufficient amounts to cause depletion of shed antigen-specific B cells bearing the idiotype or cross-reactive idiotype recognized by the induced immune response. Induction by the Id vaccine of anti-idiotypic antibodies alone are capable of inducing depletion of B cells comprising shed antigen-specific B cells. As an example of an additional mechanism of operability, and depending on the composition of the Id vaccine and the mode of vaccination, the anti-idiotypic antibody induced may bind anti-shed antigen antibody in forming immune complexes, and therefore may also play a role in the beneficial clinical outcome resulting from Id vaccination according to the present invention by competing with shed antigen for the anti-shed antigen antibody (i.e., the anti-idiotypic antibody may inhibit immune complex formation between shed antigen and anti-shed antigen antibody, and hence, reduce activation of immune effector cells by such immunostimulatory immune complexes).

The present invention also provides vaccine formulations comprising an Id vaccine for inducing an immunological response against shed antigen-specific B cells that may be present; wherein such B cells comprise a sIg having binding specificity for an epitope of shed antigen. In one embodiment, the vaccine formulation comprises one or more peptides wherein each peptide comprises an antigenic portion of the idiotype of an antibody having binding specificity for an epitope of shed antigen. When the vaccine formulation is administered in an immunologically effective amount, induced is an immune response comprising antibody against the idiotypes in the vaccine formulation (anti-idiotypic antibody). In one embodiment, the one or more peptides is administered with an adjuvant to enhance the immunogenicity of the one or more peptides, in forming a vaccine formulation. Adjuvants are known to those skilled in the art to include, but are not limited to, saponin, oil emulsions, adjuvant 65 (containing vegetable oil, mannide, monooleate and aluminum monostearate), Ribi adjuvant, alum, polyamines, pluronic polyols, mineral gels such as aluminum hydroxide, aluminum phosphate, CpG dinucleotides, and the like. In a preferred embodiment, the one or more peptides may further comprise an immunostimulatory sequence, operatively coupled to the idiotype, which may enhance the immunogenicity of the one or more peptides.

In another embodiment of the vaccine formulation for an Id vaccine according to the present invention, the vaccine formulation comprises a nucleic acid molecule (“polynucleotide”) which encodes a peptide comprising an idiotype found on shed antigen-specific B cells; or comprises a plurality of polynucleotides, each encoding a encodes a peptide comprising an idiotype found on shed antigen-specific B cells. The vaccine formulation comprising the one or more polynucleotides may be administered in a suitable delivery method of genetic immunization known to those skilled in the art to include, but not limited to, direct injection into muscle, direct injection into the ear pinna, delivery in a complex with protein carriers (e.g., atelocollagen, gelatin, collagen), encapsulated in liposomes, complexed with nucleic acid molecules comprising CpG dinucleotides, cutaneous delivery by particle bombardment (e.g., using a gene gun), in vivo infection (if used are viral vectors), and a combination thereof. It is apparent to one skilled in the art that the delivery method of a genetic vaccine may affect the immunization response. For example, DNA delivered intradermally may prime predominately type 1 T cell help; whereas gene gun delivery may DNA prime type 2 T cell help. Thus, if both types of T cell help are desired, a combination of two different methods for delivering the vaccine formulation may be desirable. When administered to an individual in an immunologically effective amount, from the vaccine formulation is produced one or more peptides in an effective amount to induce anti-idiotype antibody. The polynucleotide of a vaccine formulation may further comprise a sequence encoding an immunostimulatory sequence. For example, where the polynucleotide is an expression vector, copies of CpG dinucleotides in the vector sequence (e.g., non-coding region with respect to the peptide) may act as adjuvants facilitating the induction of an immune response against the expressed peptide. CpG dinucleotides are known in the art to comprise a stretch of DNA (e.g., of a length in a range of from about 5 base pairs to about 50 bp) containing CpG dinucleotide within a specified sequence. Exemplary CpG dinucleotides are shown as SEQ ID Nos:12 & 13. In another example, the nucleic acid sequence encoding the peptide also encodes an immunostimulatory sequence such that the peptide comprises the idiotype and further comprises the immunostimulatory sequence operatively coupled to the idiotype to optimize the desired immune response against the idiotype. A linker, as known to those skilled in the art, may be used to operatively couple a nucleic acid sequence to another nucleic acid sequence wherein the sequences encode molecules selected from the group consisting of: a V_{H} to a V_{L}, a V_{H} to an immunostimulatory sequence, a V_{L} to an immunostimulatory sequence, and a combination thereof. For example, a linker may comprises a nucleic acid sequence, such as a nucleic acid sequence encoding amino acids 115 to 129 which was used link the sequence encoding amino acids 1-114 to the sequence encoding amino acids 130-208, of SEQ ID NO:1. The polynucleotide may further comprise a sequence encoding a repressible control element, operatively linked to the nucleic acid sequence encoding the peptide comprising the idiotype, and from which can be controlled (e.g., repressed) the expression from the polynucleotide of the nucleic acid sequence encoding the peptide. Repression of expression from the polynucleotide may then be effected by the administration to the individual of an amount of repressor agent sufficient to effect the repression by interacting with the repressible control element.

### EXAMPLE 1

In this example, illustrated is (a) B cell involvement, and the specific type of immune response related thereto, promotes tumor progression and metastasis; and (b) the effect of an immunological response involving shed antigen-specific B cells, present in an immune complex-mediated disease progession of an organ-specific autoimmune disease. While the organ-specific autoimmune disease illustrated in this example is immune-complex mediated disease progression of solid, nonlymphoid tumor, it will be apparent to one skilled in the art from the following description that a similar immunological response can occur in other organ-specific autoimmune diseases in which shed antigen induces antibody having binding specificity for an epitope expressed by shed antigen. Likewise, while the antibody illustrated is an antibody having binding specificity for an epitope comprising a terminal, alpha 2,6-linked sialic acid, it will be apparent to one skilled in the art that other epitopes of shed antigen induce a similar immune response, particularly epitopes comprising terminal alpha-linked carbohydrates other than sialic acid. In that regard, some of the findings illustrated in this example as observed for individuals with immune-complex mediated disease progression of solid, nonlymphoid tumor have also been observed for individuals susceptible to (e.g., relapsing remitting multiple sclerosis) or have a chronic progressive form of multiple sclerosis (e.g., secondary progressive multiple sclerosis).

Relevant to some of the following illustrations, it is important to consider the following concept. Various strains of mice were used as a standard animal model for evaluating whether a B cell response may be involved in immune complex-mediated tumor progression, including promoting metastasis. In mice bearing shed antigen-secreting tumors, observed is similar B cell phenotype including shed antigen-specific B cells, and immune complexes containing antibody having binding specificity for an epitope of shed antigen (e.g., epitopes comprising a terminal, alpha-linked carbohydrate) as that observed in humans with solid, nonlymphoid tumor and a pro-tumor immune response. Further, mice having immune deficiencies have been accepted as a standard in vivo model for assessing therapeutic approaches (e.g., Id vaccines) to human B cells (e.g., lymphomas).

1.1 B Cells Involved in Tumor Promotion Include Those Exposed to Shed Tumor Antigen.

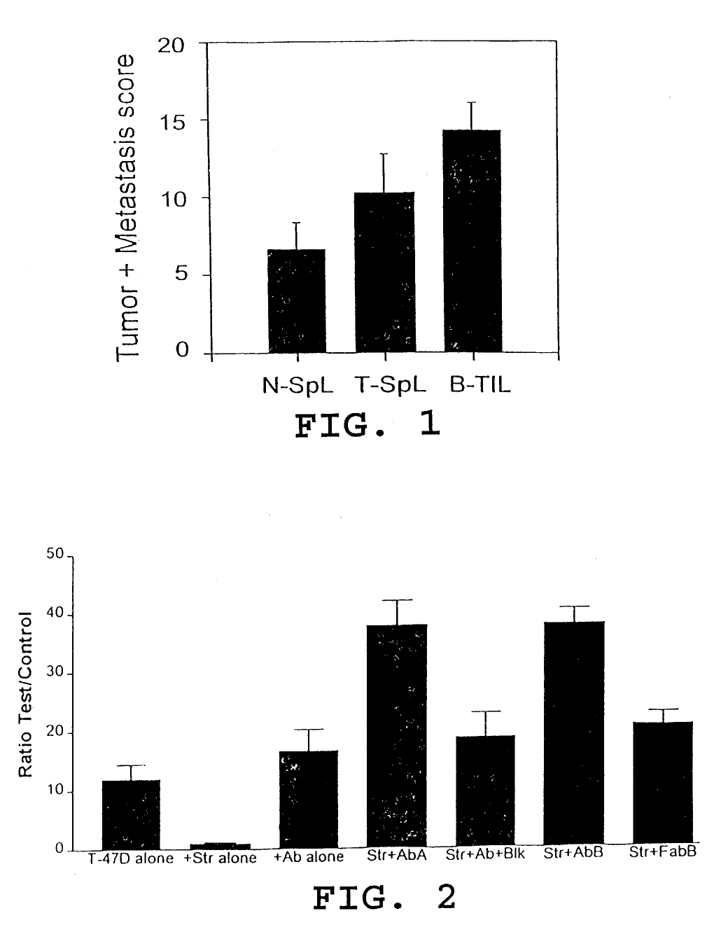

To assess whether different populations of B lymphocytes could promote growth of the tumors which produce shed antigen (e.g., shed tumor mucin) in vivo, growth of mucinous tumors in mammary glands of C3H mice was compared when the mice were injected every 2 days for a 14 day period with either B lymphocytes (50,000 cells) isolated from normal mouse spleen; B lymphocytes isolated from lymphoid tissues (e.g., spleens) of mucinous-tumor bearing mice (50,000 cells), or tumor infiltrating B lymphocytes (B-TIL; 50,000 cells) isolated from mucinous tumors of tumor bearing mice. Isolations of B lymphocytes were performed by magnetic separation methods known in the art. After the 14 day period, liver metastasis and spleen tumor growth (tumor+metastasis score) were evaluated and scored. As shown in FIG. 1, B-TIL and B lymphocytes from spleens of tumor bearing mice (“T-Spl”) each promoted statistically significant tumor growth and metastasis in vivo, whereas B lymphocytes from normal spleen (“N-Spl”) did not enhance either tumor growth or metastasis. An important conclusion that can be drawn from these results is that to gain the ability to promote tumor growth, B lymphocytes must first be exposed to tumor antigens (e.g., prior contact with shed tumor antigen).

1.2 Antibody Against Shed Antigen Epitopes Can Promote Immune Complex-mediated Disease Progression in Vivo.

Groups of nude mice and groups of SCID mice were injected with a human colon carcinoma cell line which sheds mucin having epitopes comprising terminal alpha 2,6 linked sialic acid (e.g., sTn), and Tn; and produces CEA with epitopes comprising terminal alpha 2,6 linked sialic acid. Each group was administered a series of injections of either a monoclonal antibody (mAb) having binding specificity for sTn (IgGl isotype), an anti-CEA mAb (IgGl isotype), a control isotype (IgGl) mAb not known to have any binding specificity for the tumor, and buffer (PBS). Tumor growth was then measured thirty days after receiving tumor. Table 1 shows, for each treatment group, the average tumor size as measured in millimeters±standard error of the mean (“Size”). As shown in Table 1, mice receiving anti-sTn mAb showed a significant accelerated rate of tumor growth compared to mice receiving either the control isotype mAb (“Control mAb”) or buffer (“Control”). Also, as shown in Table 1, nude mice which received anti-CEA mAb showed a significant accelerated rate of tumor growth compared to mice receiving either the control isotype mAb (“Control mAb”) or buffer (“Control”). However, for those nude mice receiving anti-CEA mAb, the accelerated rate of tumor growth was only evident after the tumor had shed CEA, and only after the nude mice developed an IgG response against the shed CEA. These results show that antibody having binding specificity for an epitope of a shed antigen can be involved in immune complex-mediated disease progression of an organ-specific autoimmune disease.

TABLE 1

| Mice | Treatment | Size |
|---|---|---|
| nude | Control | 157.9 ± 7.0 |
| | Control mAb | 94.9 ± 5.8 |
| | anti-sTn mAb | 207.8 ± 6.9 |
| | anti-CEA mAb | 195.7 ± 4.9 |
| SCID | Control | 73.1 ± 5.9 |
| | Control mAb | 70.9 ± 5.7 |
| | anti-sTn mAb | 127.2 ± 6.0 |
| | anti-CEA mAb | 47.9 ± 5.6 |

The following example is an additional illustration that antibody having binding specificity for an epitope of a shed antigen can be involved in immune complex-mediated disease progression of an organ-specific autoimmune disease. Groups of SCID mice were injected subcutaneously with a human colon carcinoma cell line which sheds mucin having epitopes comprising terminal, alpha 2,6 linked sialic acid (e.g., sTn). Additionally, some groups were co-injected with either a hybridoma secreting anti-sTn mAb (“hybridoma A”), or a hybridoma secreting a control isotype (IgGl) mAb not known to have any binding specificity for the tumor (“hybridoma B”). Survival (as measured by the percent which survived 100 days after receiving tumor), angiogenesis (number of vessels per mm²), and metastasis (as measured by the number of metastases per liver at 40 days after receiving the tumor) were compared between mice receiving tumor alone (“Tumor”), mice receiving tumor plus hybridoma A (“Tumor+hybridoma A”) and mice receiving tumor plus hybridoma B (“Tumor+hybridoma B”). As shown in Table 2, mice receiving tumor+hybridoma A (secreting an antibody having binding specificity for an epitope comprising a terminal, alpha 2,6 linked sialic acid) showed a significantly lower survival rate, significantly increased angiogenesis, and significantly more metastases than mice receiving either tumor alone or tumor+hybridoma B (secreting a control isotype mAb). These results show that antibody having binding specificity for an epitope of shed antigen (as illustrated by antibody having binding specificity for an epitope comprising a terminal alpha 2,6 linked sialic acid) can be involved in immune complex-mediated disease progression of an organ-specific autoimmune disease.

TABLE 2

| Treatment | Survival | Angiogenesis | Metastasis |
|---|---|---|---|
| tumor | 20% | 13 ± 3 | 0 |
| tumor + | 0% | 146 ± 23 | 18 ± 4.8 |
| hybridoma A | | | |
| tumor + | 60% | 3 ± 0.1 | 0 |
| hybridoma B | | | |

1.3 A Mechanism by Which Immune Complexes Comprising Antibody Having Binding Specificity for an Epitope of Shed Antigen Can Promote Immune Complex-mediated Disease Progression.

This example illustrates only one of several mechanisms which we discovered by which immune complexes comprising anti-shed antigen antibody and shed antigen promote immune complex-mediated disease progression of an organ-specific autoimmune disease. The illustrated mechanism involves the binding by these immune complexes to cell surface receptors, such as FcγRI on FcγRI-expressing immune effector cells (one or more of neutrophils, macrophages, endothelial cells, astrocytes, microglia, and the like), with resultant crosslinking of the bound receptors. In this illustrative example, an in vitro tumor cell invasion assay was used which included mucin-secreting human tumor cell line T-47D, a Boyden chamber, and a commercially available basement membrane matrix preparation (“matrix”). In this assay, tested was the ability of the immune complexes to activate immune effector cells to degrade the tissue matrix, as assessed by the ability of tumor cells (2×10⁴ cells) to migrate through the matrix (“invasion”) in the following conditions: matrix alone; matrix containing stromal cells (mixture of granulocytes and macrophages; 2×10⁵ cells; “Str”); matrix in the presence of either of two different antibodies (“AbA”, and “AbB”) having binding specificity for a shed tumor mucin epitope comprising a terminal, alpha 2,6-linked sialic acid (e.g., anti-sTn mAb; IgGl; 0.06 μg); matrix containing stromal cells in the presence of anti-sTn mAb; and matrix containing stromal cells in the presence of Fab fragments of AbB. The plates were incubated at 37° C. in 5% CO₂, and fresh media (with or without antibody/antibody fragment, depending on the condition) was substituted every 24 hours. Invasion was measured by counting the number of tumor cells per well which migrated to the bottom of the chamber after 48 to 72 hours. FIG. 2 shows that the maximum invasion through the matrix was observed when the shed tumor antigen-secreting tumor cells were incubated in the presence of stromal cells and either of two anti-sTn mAb tested (“Str+AbA”; and “Str+AbB”), as compared to tumor cells alone (“T-47D alone”), or stromal cells (“+Str alone”), or anti-sTn mAb (“+Ab alone), or stromal cells in the presence of Fab fragments of the anti-sTn mAb (Str+FabB). These results are further evidence that shed antigen can complex with anti-shed antigen antibody in forming immune complexes that can activate immune effector cells to secrete molecules (e.g., one or more of tissue degrading enzymes, cytokines, oxygen free radicals) that promote disease progression such as by tissue degradation. The involvement of immune complexes, as opposed to the action of antibody alone, was confirmed by using a tumor cell which did not produce sTn-containing shed tumor antigen; i.e., when such tumor cells were incubated in the presence of stromal cells and anti-sTn mAb, there was no increase in tumor invasion as compared to the control values. Additionally, the evidence suggests that immune complexes comprising anti-shed antigen antibody complexed to shed antigen can have a threshold level for spacing and number of antibody molecules necessary for receptor (e.g., FcγRI) crosslinking on immune effector cells.

1.4 Depleting B Cells (Mature B Cells and/or Memory B Cells) Can Interrupt B Cell Involvement in a Pro-tumor Response in Vivo, Thereby Affecting Tumor Progression.

In one illustration of this example, fifty three C3H mice were injected intrasplenically with 10⁶ Met 129 tumor cells (high mucin-producing mammary carcinoma cells). The injected mice were then divided into two treatment groups. One group of 28 mice was injected with an irrelevant (not directed against any specific mouse antigen) goat IgG antibody (170 μg per injection) at days 5, 7, and 9 following tumor challenge. A second group consisted of 25 mice injected with goat anti-mouse IgG (170 μg per injection) at days 5, 7, and 9 following tumor challenge. The goat anti-mouse IgG was used to deplete the C3H mice of their B cells, thereby interrupting the host B cell-mediated pro-tumor immune response. At 22 days following tumor challenge, the two groups of mice were analyzed for primary tumor growth in the spleen (Table 3, “Tumor”), metastasis to the liver (Table 3, “Liver Met.”), and extra-regional metastasis (abdominal lymph nodes; Table 3, “Extra-R Met.”). Table 3 shows that there is a statistically significant reduction in the incidence of metastasis in the B cell-depleted mice (“Anti-IgG”) as compared to the control group receiving irrelevant IgG (“Goat-IgG”).

TABLE 3

| Observed | Goat-IgG Control | Anti-IgG |
|---|---|---|
| Tumor | 8 of 8 | 6 of 6 |
| Liver Met. | 5 of 8 | 0 of 6 |
| Extra-R Met. | 6 of 8 | 0 of 6 |

In summary, the results illustrated in Table 3 further support the finding that B cell depletion, such as depletion of shed antigen-specific B cells, can inhibit an immune complex-mediated disease progression of an organ-specific autoimmmune disease.

Similar results have been observed in humans. More specifically, administered to individuals having advanced cancer (Stage IV, solid, nonlymphoid tumor) and a pro-tumor immune response was an immunotherapeutic composition comprising a chimeric anti-CD20 mAb, in a therapeutically effective amount to deplete B cells. To each individual was administered, by intravenous infusion, an initial dosage of 200 mg of the immunotherapeutic composition; and then administered were at least two additional infusions, with each additional infusin spaced apart by four weeks from the previous infusion. The rate of infusion was dependent on how the individual tolerated infusion, the treating physician's judgment, drug manufacturer's instructions, and lack of side effects. Two treated individuals showed a clinical benefit (e.g., reduction in the size and number of metastases) concomitant with a reduction in shed antigen-specific B cells. However, the treatment is not as specific as desired, since some B cells not comprising shed-antigen specific B cells were also depleted as a side effect of treatment.

### EXAMPLE 2

The evidence presented in Example 1 illustrates that shed antigens can be produced in immune complex-mediated disease progression of an organ-specific autoimmune disease; that the shed antigen may comprise one or more epitopes capable of inducing a humoral immune response that results in the production of antibody having binding specificity for shed antigen; that this anti-shed antigen antibody can complex to shed antigen in forming immune complexes; that such immune complexes can have a threshold level for spacing and number of antibody molecules necessary for receptor (e.g., FcγRI) crosslinking on immune effector cells; and that a biological effect, induced by such crosslinking, can act to promote immune complex-mediated disease progression of an organ-specific autoimmune disease. From these results, it is clear that there can be a chronic activation of B cells by shed antigen. In that regard, shed antigen can be presented to newly recruited (naïve) B cells which may result in the activation of such B cells to become shed antigen-specific B cells. Hence, shed antigen-specific B cells can have, as part of their surface Ig, an idiotype that represents a specific antigen that can generally be distinguished from idiotypes present on a majority of B cells not related to an organ-specific autoimmune disease process (in noting that there may be a minor proportion of B cells in an individual which have an idiotype the same as or cross-reactive to the sIg of shed antigen-specific B cells; e.g., as a result from induction by bacterial or food-related carbohydrates). The presence of such a chronic activation of shed antigen-specific B cells has been confirmed by immunhistochemical staining of biopsied tissues involved in immune complex-mediated disease progression from humans having an organ-specific autoimmune disease. Accordingly, the idiotypes of antibodies having binding specificity for epitopes of shed antigens may be used to generate vaccine formulations comprising Id vaccines for inducing an immunologically effective immune response to cause depletion of shed antigen-specific B cells.

In one embodiment, a vaccine formulation according to the present invention comprises one or more peptides. Each peptide comprising the vaccine formulation may further comprise an immunostimulatory sequence to which it is operatively coupled. The vaccine formulation may further comprise a pharmaceutically acceptable carrier. Typically, such carriers are sterile, pyrogen free liquid media (e.g., water, salt solution, buffer, and the like), emulsions, or gels which are suitable for introducing a vaccine formulation into an individual. A peptide comprises an idiotype of an antibody having binding specificity for an epitope of shed antigen. In a preferred embodiment, the epitope comprises a terminal alpha-linked carbohydrate; and in a more preferred embodiment, the epitope comprises a terminal, alpha 2,6-linked sialic acid. In a preferred embodiment, the peptide comprises a V_{H} region linked to a V_{L} region. Illustrative examples of the peptide, which may be used in a vaccine formulation in an Id vaccine for immunization against shed antigen-specific B cells, include, but are not limited to, SEQ ID NO:1, SEQ ID NO:2, SEQ ID NO:3, SEQ ID NO:4, SEQ ID NO:5, SEQ ID NO:6, SEQ ID NO:7, SEQ ID NO:9, and a combination thereof. As an illustrative example, and in preferred embodiment, an immunostimulatory sequence which may be operatively coupled to the peptide (e.g., any one of SEQ ID NOs: 1-9) comprises SEQ ID NO:10; and as illustrated by SEQ ID NO:11, which is a vaccine formulation comprising a peptide comprising SEQ ID NO:1 operatively coupled to SEQ ID NO:10.

In a preferred embodiment, the vaccine formulation comprises one or a plurality of polynucleotides which, when administered to an individual to be immunized, is taken up by cells of the individual; and wherein the one or more polynucleotides may remain in the cells as functioning episomal molecules or may integrate into the cells' chromosomal DNA. Preferably, the one or more polynucleotides may be introduced into cells, and maintained as separate genetic material, such as in the form of a plasmid. The necessary elements of a polynucleotide comprising the vaccine formulation include a nucleotide sequence that encodes a peptide according to the present invention, and regulatory elements necessary for the expression of the sequence in the cells of the immunized individual. The regulatory elements are operably linked to the nucleotide sequence to enable expression from the sequence. Such regulatory elements are known to those skilled in the art to incude a promoter, initiation codon, a stop codon, and a polyadenylation signal, all of which are functional in the vaccinated individual. Various promoters to practice the polynucleotide according to the present invention, and known to those who construct genetic vaccines for humans, include but are not limited to: human hemoglobin promoter, human muscle creatinine promoter, human actin promoter, human myosin promoter, Epstein Barr virus (EBV) promoter, cytomegalovirus (CMV) promoter, Moloney virus promoter, mouse mammary tumor virus (MMTV) promoter, human immunodeficiency virus long terminal repeat (HIV-LTR) promoter, and Rous sarcoma virus (RSV) LTR promoter. Likewise, the regulatory elements may additionally include an enhancer, and may be selected from enhancers of gene expression for the same genes listed as sources for promoter sequences. Various polyadenylation signals known to those who construct genetic vaccines for humans, include but are not limited to: an SV40 polyadenylation signal, a beta-globin polyadenylation signals, a LTR polyadenylation signal, and a synthetic polyadenylation signal. In a preferred embodiment, the polynucleotide is in a plasmid vector which, when administered to an individual to be immunized, is taken up by cells of the individual and remains extrachromosomally as a separate genetic element. To maintain the plasmid vector as an extrachromosomal element inside the cell, and to produce multiple copies of the vector in the cell, the vector may be constructed to have a mammalian origin of replication such as an SV40 origin of replication or an EBV origin of replication. Expression vectors that are known to those skilled in the art as useful for genetic immunization include, but are not limited to, pCDNA3, pCMV, pRSV, and episomal vectors (see, e.g., U.S. Pat. No. 5,624,820, herein incorporated by reference).

In a preferred embodiment, a polynucleotide comprising a vaccine formulation according to the present invention was produced by isolating mRNA from cells producing antibody having binding specificity for an epitope of shed antigen. The mRNA was reversed transcribed to cDNA using a commerical kit for reverse transcription. From the mRNA was amplified the Fv heavy chain and the Fv light chain, and linked with a sequence encoding a (Gly₄Ser)₃ linker using a commercial kit. The resulting sequence, encoding a peptide comprising V_{H}-V_{L} (SEQ ID NO:1) was then cloned into a commercially available vector (pCR2.1). The sequence was then subcloned into a mammalian expression vector (pcDNA3.1) using restriction enzyme digestion (Eco RI and Xho I) and ligation methods known in the art. The orientation of the sequence encoding V_{H}-V_{L} in the resultant polynucleotide was then confirmed. To produce another polynucleotide comprising a vaccine formulation, the polynucleotide was further modified to encode an immunostimulatory sequence. In that regard, a sequence encoding tetanus toxin fragment-C (FrC; see, e.g., SEQ ID NO:10, amino acids 2-453) was cloned (e.g., by restriction with Xho I, and subsequent ligation) into the polynucleotide in frame with the sequence encoding V_{H}-V_{L} to result in a polynucleotide comprising an insert for expression, wherein the insert comprised a nucleic acid sequence encoding V_{H}-V_{L}-FrC. As shown by SEQ ID NOs:10 & 11, the first two amino acids of FrC represent a linker comprising two amino acids encoded by a restriction enzyme cloning site, followed by 451 amino acids of FrC. It will be apparent to one skilled in the art from this illustration that a peptide according to the present invention (including any one of SEQ ID NOs: 1-9) may be recombinantly produced with FrC fused to the carboxy terminus of the peptide resulting in a vaccine formulation. While it is a preferred embodiment that the FrC be operatively coupled at the carboxy terminus of the peptide, it will also be apparent to one skilled in the art that FrC may instead be operatively coupled to the amino terminus of the peptide.

It may be desirable to modulate in an immunized individual the induction of an immune response induced by the vaccine formulation comprising one or more polynucleotides. In that regard, in one embodiment of the vaccine formulation each polynucleotide further comprises a tetracycline (Tc)-controlled gene expression system that quantitatively controls gene expression in mammalian cells. For example, the polynucleotide and regulatory elements may be operatively linked to a Tc-controlled activator which, in the absence of tetracycline, induces expression of the operatively linked gene. The Tc-controlled activator may comprise a tetracycline repressor fused to a transcription activation domain that activates transcription in mammalian cells. By adding tetracycline, subsequent gene expression is inhibited; which expression vector system is particlarly adapted for gene immunization (see, e.g., vector VR1370, ATCC deposit accession number ATCC 97467). Thus, a eukaryotic expression plasmid vector is provided in which the polynucleotide is operatively linked to, and placed under the control of a tetracycline-controlled activator-responsive promoter (the tetracycline operator sequence upstream of a minimal promoter). Also operatively linked to the tetracycline-controlled activator-responsive promoter is a tetracycline-controlled activator comprising a tetracycline repressor protein fused to a transcription activation domain. By the addition of a pharmaceutically effective amount of tetracycline to a individual immunized with vaccine formulation comprising this vector containing the polynucleotide under tetracycline control, the DNA binding activity of the tetracycline-controlled activator is suppressed by the bound tetracycline, thereby resulting in repression of expression of the polynucleotide encoding the idiotype.

### EXAMPLE 3

In a method according to the present invention, produced are nucleotide sequences (from which can be deduced amino acid sequences) of idiotypes of shed antigen-specific B cells involved in an organ-specific autoimmune disease. Sequences, encoding idiotypes of shed antigen-specific B cells involved in an organ-specific autoimmune disease, can be identified using standard techniques well known in the art. A method for producing a vaccine formulation comprising a plurality of polynucleotides comprising idiotypes of shed antigen-specific B cells comprises: isolating B cells from a body fluid or tissue containing shed-antigen specific B cells; isolating mRNA from the B cells; reverse transcribing the mRNA to cDNA; amplifying sequences encoding V_{H} and sequences encoding V_{L} from the cDNA; linking sequences encoding V_{H} with sequences encoding V_{L} with a sequence encoding a linker in producing a plurality of V_{H}-V_{L} sequences; and cloning the plurality of V_{H}-V_{L} sequences into a mammalian expression vector for genetic immunization in forming a plurality of polynucleotides; wherein the plurality of polynucleotides comprise the vaccine formulation.

For example, from an individual having an organ-specific autoimmune disease (including having the immune response which could lead to development of the organ-specific autoimmune disease) can be isolated shed antigen-specific B cells. Shed antigen-specific B cells may be isolated from one or more of: a body fluid draining the site of the organ-specific autoimmune disease (e.g., blood or effluent for solid, nonlymphoid tumor, cerebrospinal fluid for multiple sclerosis), tissues comprising lymphoid tissue proximal to or in the site of the organ-specific autoimmune disease process, and tissue showing pathology as a result of the organ-specific autoimmune disease process (e.g., the anatomical site of the organ-specific autoimmune disease). In one illustrative embodiment of this method of the present invention, and where the organ-specific autoimmune disease comprises a solid, nonlymphoid tumor a pro-tumor immune response, available tissue from which nucleic acid material of shed antigen-specific B cells may be isolated or obtained includes one or more of lymphoid tissues containing shed tumor antigen (generally located proximal to the tumor), biopsy of tumor tissue containing infiltrating B cells, peripheral blood, and aspirates of fluid associated with tumor (and including tissue aspirates). The tissue may be directly processed to obtain the nucleic acid material from the shed antigen-specific B cells contained therein; or may be used as a source from which can be isolated shed antigen-specific B cells, and wherein the nucleic acid material can be isolated or obtained from the isolated shed antigen-specific B cells.

For instance, tissue may be processed using mechanical and enzymatic (e.g., collagenase, DNase) treatment to dissociate the tissue into a cell suspension. From this cell suspension, or from cells of a cell-containing body fluid (e.g., peripheral blood) can be prepared RNA and cDNA using methods known in the art, and V_{H} gene sequences or V_{H} and V_{L} gene sequences may be amplified using standard nucleic acid amplification techniques and known primers. Alternatively, from cells can be isolated mononuclear cells by layering the cells on a density gradient medium (commercially available) followed by centrifugation. The fraction comprising mononuclear cells is harvested. B cells may be enriched, such as by immunomagnetic selection using methods known in the art (e.g., with magnetic beads coated with anti-CD19 antibody). In yet another alternative, the mononuclear cells may be enriched for B cells by immunomagnetic selection for plasma cells, which are then either immortalized by Epstein-Barr virus-transformation, or fused to myeloma cells using methods known in the art (e.g., PEG fusion, or electrofusion). From these cells may be prepared cDNA; and V_{H} gene sequences, or V_{H} and V_{L} gene sequences may be amplified using standard nucleic acid amplification techniques and known primers.

For example, primers useful in amplifying human V_{H} gene sequences from cDNA include SEQ ID NOs:14-19 (non-degenerate primers for each of the six human V_{H} gene families), with a 3′ antisense primer chosen from SEQ ID NO:20 (FR3 sequence) or SEQ ID NO:21 (J region sequence). From analysis of the amplification process, amplified products may be sequenced using methods and primers known in the art (e.g., dideoxy chain termination). The resultant sequences characteristic of involvement in the organ-specific autoimmune disease can be readily recognized by one or more of: its frequency of occurrence (e.g., repeated occurrence among various B cell clones analyzed); by comparison with control normal tissue or control reactive tissue (wherein lack of occurrence of the sequence in control tissues is suggestive of involvement related to the organ-specific autoimmune disease process); and by comparison (for identity using standard comparison computer programs known in the art) with Id sequences of antibodies known to bind an eptitope on shed antigen (e.g., SEQ ID NOs: 1-9). Regarding comparisons, it is known that B cells have a significant tendency to express V_{H} genes of sequence similarity when the cognate antigen has the same or similar carbohydrate structure (“clonal relationship”; see, e..g., Zenita et al., 1990, J. Immunol. 144:4442-4451); i.e., amino acid sequences that are well adapted to binding a an epitope comprising a terminal carbohydrate of a particular linkage frequently share substantial nucleic acid sequence similarity, which allows them to be identified by such comparison to known sequences (e.g., encoding any one of SEQ ID NOs:1-9). After identifying a nucleic acid sequence which is representative of that which encodes a peptide having binding specificity for an epitope of shed antigen, a peptide can be deduced from the sequence and the deduced peptide synthesized using methods known in the art. Alternatively, the nucleic acid sequence encoding the peptide may be cloned into a mammalian expression vector in forming a polynucleotide.

Various expression vectors have been constructed to contain a nucleic acid sequence which encodes a peptide selected from the group consisting of SEQ ID NOs:1-9 for recombinant expression of the respective peptide from a transformed host cell. For example, inserted into and operatively linked to a vector containing a T7 promoter, lacI coding sequence, multiple cloning sites, a His-Tag sequence, and various other sequences necessary for replication and expression in Escherichia coli, was the sequence encoding a peptide to be expressed. A culture of E. coli, transformed with the resultant vector, was induced with an inducing agent, cultured, collected by centrifugation, lysed, and the lysate was placed over a nickel affinity column to purify the recombinantly producued peptide via its His-Tag squence. As will be apparent to those skilled in the art, other commercially available vectors may be used for expressing the peptide of interest. One or more purification steps, using conventional techniques known to those skilled in the art (e.g., one or more of ammonium sulfate precipitation, gel filtration, ion exchange chromatography, affinity chromatography, and the like) may be utilized in order to obtain the recombiantly produced peptide sufficiently pure to be acceptable for administration to an individual.

### EXAMPLE 4

In this example are illustrated various embodiments of a method for inducing an immune response reactive with idiotypes present on shed antigen-specific B cells in an individual by administering an immunologically effective amount of a vaccine formulation according to the present invention. One goal of the method is to induce an immunological response against shed antigen-specific B cells by inducing an immune response comprising anti-idiotype antibody.

In a first embodiment, the vaccine formulation comprises one or more peptides according to the present invention, and further comprises: an immunostimulatory sequence operatively coupled to each peptide, an adjuvant, or a combination thereof. The vaccine formulation may further comprise a pharmaceutically acceptable carrier. An immunologically effective amount of the vaccine formulation may be administered directly to the individual using any methods of delivery known to those in the art. For example, the vaccine formulation may be administered parenterally, intramuscularly, intraperitoneally, transdermally, subcutaneously, and the like. The exact amount of the peptide in the vaccine formulation to achieve an immunologically effective amount will vary from individual to individual depending on such factors as age, weight, general health, severity of any organ-specific autoimune disease process; the particular peptide to be used in the vaccine formulation; and the route of administration. An appropriate amount of the peptide and the vaccine formulation may be determined by one skilled in the art using only routine experimentation given the teachings herein. Generally, an approximate dosage for administration of peptide vaccines is in the range of about 5 μg to about 1000 μg; and more preferably is in the range of from about 50 μg to about 500 μg. The number of doses (e.g., the number of times the vaccine formulation may be administered to the individual in immunizing the individual) may vary, and is generally in the range of 1 to 10. Thus, in one embodiment, the individual to be immunized receives more than one dose (a series of doses) of the vaccine formulation. When receiving more than one dose, each dose may differ or be the same in terms of one or more of amount, and route of administration. Additionally, when receiving more than one dose, the time period between each dose may be established by one skilled in the art, but generally may be a matter of weeks, and more preferably in a range of 2 to 10 weeks.

In another embodiment, the method according to the present invention comprises administering to the individual an immunologically effective amount of a vaccine formulation comprising one or more polynucleotides, wherein a polynucleotide comprises a mammalian expression vector having operatively cloned therein for expression a nucleic acid sequence encoding an idiotypic determinant comprising an antigenic determinant present in the unique antigen recognition site of sIg on shed antigen-specific B cells. In a preferred embodiment, the polynucleotide encodes a peptide comprising the idiotype and further comprising an immunostimulatory sequence for expression in mammalian cells. The nucleic acid sequence encodes a peptide comprising the idiotypic determinant which is expressed in the individual's cells into which it is introduced, and is then presented (e.g., such as by secretion from the cells or presented by antigen presenting cells) to the immune system of the individual in inducing the immune response. The vaccine formulation may further comprise a pharmaceutically acceptable carrier, an adjuvant (e.g., cationic liposome complex, protein carrier, CpG dinucleotides, and the like) or a combination thereof. Once the polynucleotide is introduced into cells following administration, cells containing the polynucleotide may then produce the idiotype vaccine for subsequent exposure to the immune system of the immunized individual. Alternatively, cells of the individual to be immunized are treated ex vivo with the polynucleotide (e.g., by methods known in the art for introducing nucleic acid molecules into cells, such as by transfection, electroporation, particle bombardment, and the like) so that the polynucleotide is introduced into the treated cells, and then cells containing the polynucleotide are re-introduced back into the patient.

The vaccine formulation may be administered directly to the individual using any methods of delivery of and vaccination with polynucleotides known to those in the art. Preferably, the vaccine formulation may be administered intramuscularly, subcutaneously, and by injection into the ear pinna. The exact amount of the one or more polynucleotides in the vaccine formulation to achieve an immunologically effective amount will vary from individual to individual depending on such factors as age, weight, general health, severity of any organ-specific autoimune disease process; the particular polynucleotide to be used in the vaccine formulation; the shed antigen inducing the humoral immune response; and the route of administration. An appropriate amount of the vaccine formulation may be determined by one skilled in the art using only routine experimentation given the teachings herein. Generally, an approximate dosage for administration of polynucleotlde vaccines is in the range of about 0.15 μg to about 1000 μg of the nucleic acid material comprising the vaccine formulation; and more preferably is in the range of from about 50 μg to about 250 μg. The number of doses (e.g., the number of times the vaccine formulation may be administered to the individual in immunizing the individual) may vary, and is generally in the range of 1 to 10. Thus, in one embodiment, the individual to be immunized receives more than one dose (a series of doses) of the vaccine formulation. When receiving more than one dose, each dose may differ or be the same in terms of one or more of amount, and route of administration. Additionally, when receiving more than one dose, the time period between each dose may be established by one skilled in the art, but generally may be a matter of weeks, and more preferably in a range of 2 to 10 weeks.

As an illustration of an embodiment of the method according to the present invention, administered to an individual was an immunologically effective amount of a vaccine formulation comprising a polynucleotide encoding, and for expressing, a peptide operatively coupled to an immunostimulatory sequence. Vaccine formulations, each comprising a polynucleotide comprising a plasmid expression vector for genetic immunization of mammals, was constructed as previously described in Example 2 herein. A first vaccine formulation comprised a polynucleotide which encodes for a peptide comprising SEQ ID NO:1 (e.g., V_{H}-V_{L}). A second vaccine formulation comprised a polynucleotide which encodes for a peptide comprising SEQ ID NO:11 (e.g., V_{H}-V_{L}-ISS). C3H mice were first injected with 1 million Met 129 tumor cells to initiate tumor growth. Met 129 tumor produces shed antigen comprising shed tumor mucin. Three days after tumor cell injection, to separate groups of mice were administered 100 μl (of a 500 μg DNA/ml of pharmaceutically acceptable carrier) of the either of the vaccine formulation by intramuscular injection. Two additional injections were given at three day increments. Development of tumor was monitored daily by measuring tumor size. Development of tumor was then compared in those mice immunized with a vaccine formulation according to present invention, and mice which received either control plasmid (plasmid without the insert encodng the peptide), or a buffer (sham-immunization). Ongoing studies indicates that mice receiving control plasmid or sham-immunized developd tumor. In contrast, and at the present time of evaluation, mice immunized with either vaccine formulation according to present invention failed to develop tumor. This result suggests that the vaccine formulations induced an immune response which inhibited an immune complex-mediated disease process for promoting tumor progression.

The foregoing description of the specific embodiments of the present invention have been described in detail for purposes of illustration. In view of the descriptions and illustrations, others skilled in the art can, by applying, current knowledge, readily modify and/or adapt the present invention for various applications without departing from the basic concept, and therefore such modifications and/or adaptations are intended to be within the meaning and scope of the appended claims.

What is claimed is:

1. A method for inducing an immune response reactive with idiotypes present on shed antigen-specific B cells that may be present in an individual, the method comprising:
administering to the individual an immunologically effective amount of a vaccine formulation comprising one or more polynucleotides;
wherein a polynucleotide of the vaccine formulation comprises a mammalian expression vector having operatively cloned therein for expression a nucleic acid sequence encoding an idiotypic determinant in an antigen recognition site of surface immunoglobulin on shed antigen-specific..B cells;
wherein the nucleic acid sequence of a. polynucleotide encodes a peptide comprising the idiotypic determinant which is expressed in the individual's cells into which it is introduced; and
wherein peptide expressed by the individual's cells is then presented to the immune system of the individual in inducing the immune response.

2. The method according to claim 1, wherein the peptide comprises a V_{H} sequence linked to a V_{L} sequence using a linker.

3. The method according to claim 1, wherein the peptide encoded by the nucleic acid sequence of a polypeptide further comprises an immunostimulatory sequence.

4. The method according to claim 1, wherein the vaccine formulation further comprises a pharmaceutically acceptable carrier.

5. The method according to claim 1, wherein the vaccine formulation further comprises an adjuvant.

6. The method according to claim 1, wherein the vaccine formulation further comprises a pharmaceutically acceptable carrier and adjuvant.

7. The method according to claim 1, wherein surface immunoglobulin on the shed antigen-specific B cells has binding specificity for an epitope comprising a terminal alpha-linked carbohydrate.

8. The method according to claim 7, wherein the terminal alpha-linked carbohydrate comprises a terminal alpha 2,6-linked sialic acid.

9. The method according to claim 1, wherein the immune response causes a depletion of shed antigen specific B cells.

10. The method according to claim 1, wherein the immune response comprises induction of anti-idiotypic antibody.

11. A method for inducing an immune response reactive with idiotypes present on shed antigen-specific B cells that may be present in an individual, the method comprising:
administering to the individual an immunologically effective amount of a vaccine formulation comprising one or more peptides;
wherein a peptide of the vaccine formulation comprises an idiotypic determinant in an antigen recognition site of surface immunoglobulin on shed antigen-specific B cells; and
wherein peptide is then presented to the immune system of the individual in inducing the immune response.

12. The method according to claim 11, wherein a peptide of the vacccine formulation comprises a V_{H} sequence linked to a V_{L} sequence using a linker.

13. The method according to claim 11, wherein the peptide further comprises an immunostimulatory sequence operatively coupled thereto.

14. The method according to claim 11, wherein the vaccine formulation further comprises a pharmaceutically acceptable carrier.

15. The method according to claim 11, wherein the vaccine formulation further comprises an adjuvant.

16. The method according to claim 11, wherein the vaccine formulation further comprises a pharmaceutically acceptable carrier and adjuvant.

17. The method according to claim 11, wherein surface immunoglobulin on the shed antigen-specific B cells has binding specificity for an epitope comprising a terminal alpha-linked carbohydrate.

18. The method according to claim 17, wherein the terminal alpha-linked carbohydrate comprises a terminal alpha 2,6-linked sialic acid.

19. The method according to claim 11, wherein the immune response causes a depletion of shed antigen specific B cells.

20. The method according to claim 11, wherein the immune response comprises induction of anti-idiotypic antibody.

21. A method for producing a vaccine formulation comprising a plurality of polynucleotides comprising idiotypes of shed antigen-specific B cells, the method comprising: isolating B cells from a body fluid or tissue containing shed-antigen specific B cells; isolating mRNA from the B cells; reverse transcribing the mRNA to cDNA; amplifying sequences encoding V_{H} and sequences encoding V_{L} from the cDNA; linking sequences encoding V_{H} with sequences encoding V_{L} with a sequence encoding a linker in producing a plurality of V_{H}-V_{L} sequences; and cloning the plurality of V_{H}-V_{L} sequences into a mammalian expression vector for genetic immunization in forming a plurality of polynucleotides; wherein the plurality of polynucleotides comprise the vaccine formulation.

22. A vaccine formulation, for inducing an immune response reactive with idiotypes present on shed antigen-specific B cells, comprising:
one or more polynucleotides, wherein a polynucleotide of the vaccine formulation comprises a mammalian expression vector having operatively cloned therein for expression a nucleic acid sequence encoding a peptide comprising an idiotypic determinant in an antigen recognition site of surface immunoglobulin on shed antigen-specific B cells.

23. The vaccine formulation according to claim 22, wherein a peptide encoded by a polynucleotide of the vacccine formulation comprises a V_{H} sequence linked to a V_{L} sequence.

24. The vaccine formulation according to claim 22, wherein a peptide encoded by a polynucleotide of the vaccine formulation further comprises an immunostimulatory sequence operatively coupled thereto.

25. The vaccine formulation according to claim 23, wherein a peptide encoded by a polynucleotide of the vaccine formulation further comprises an immunostimulatory sequence operatively coupled thereto.

26. The vaccine formulation according to claim 22, wherein surface immunoglobulin on the shed antigen-specific B cells has binding specificity for an epitope comprising a terminal alpha-linked carbohydrate.

27. The vaccine formulation according to claim 26, wherein the terminal alpha-linked carbohydrate comprises a terminal alpha 2,6-linked sialic acid.

28. A vaccine formulation, for inducing an immune response reactive with idiotypes present on shed antigen-specific B cells, comprising one or more peptides; wherein a peptide of the vaccine formulation comprises an idiotypic determinant in an antigen recognition site of surface immunoglobulin on shed antigen-specific B cells.

29. The vaccine formulation according to claim 28, wherein a peptide of the vacccine formulation comprises a V_{H} sequence linked to a V_{L} sequence.

30. The vaccine formulation according to claim 28, wherein a peptide of the vaccine formulation further comprises an immunostimulatory sequence operatively coupled thereto.

31. The vaccine formulation according to claim 29, wherein a peptide of the vaccine formulation further comprises an immunostimulatory sequence operatively coupled thereto.

32. The vaccine formulation according to claim 28, wherein surface immunoglobulin on the shed antigen-specific B cells has binding specificity for an epitope comprising a terminal alpha-linked carbohydrate.

33. The vaccine formulation according to claim 32, wherein the terminal alpha-linked carbohydrate comprises a terminal alpha 2,6-linked sialic acid.
